SEQUENCE LISTING (6 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 3 - protein, 449 aa, Artificial
  Synthetic heavy chain sequence
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, Artificial
  Synthetic light chain sequence
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial
  Synthetic eavy chain sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 214 aa, Artificial [sequence omitted]

(19) United States
(12) Patent Application Publication
(10) Pub. No.: US 2024/0002519 A1
(43) Pub. Date: Jan. 4, 2024

(54) USE OF IL-6 INHIBITORS FOR THE TREATMENT OF ACUTE CHEST SYNDROME IN PATIENTS SUFFERING FROM SICKLE CELL DISEASE

(71) Applicants: INSERM (INSTITUT NATIONAL DE LA SANTÉ ET DE LA RECHERCHE MÉDICALE), Paris (FR); FONDATION IMAGINE, Paris (FR); ASSISTANCE PUBLIQUE-HÔPITAUX DE PARIS (APHP), Paris (FR); UNIVERSITÉ PARIS CITÉ, Paris (FR)

(72) Inventors: Slimane ALLALI, Paris (FR); Olivier HERMINE, Paris (FR); Thaigo TROVATI MACIEL, Paris (FR); Rachel RIGNAULT-BRICARD, Paris (FR)

(21) Appl. No.: 18/251,944

(22) PCT Filed: Nov. 4, 2021
(86) PCT No.: PCT/EP2021/080584

§ 371 Date: May 5, 2023

(30) Foreign Application Priority Data
Nov. 5, 2020 (EP) 20306334.2

(51) Int. Cl. C07K 16/28; C07K 16/24; C12N 15/113

(52) U.S. Cl. CPC C07K 16/2866; A61K 2039/505; C12N 15/1136; C07K 16/248

(57) ABSTRACT

Acute chest syndrome (ACS) is a common and potentially lethal form of acute lung injury in sickle cell disease (SCD). Because pathophysiology remains unclear, therapeutic options are limited to supportive care with empiric antibiotics and red cell transfusion in case of aggravation. A role of inflammation mediated by endothelial and immune cells has been suspected but the levels of pro-inflammatory cytokines and chemokines in the lungs during ACS have not yet been investigated. Here the inventors report dramatically high levels of IL-6, unlike IL-1β and TNF-α, in the sputum from SCD children during ACS (n=12) compared with non-ACS sputum (n=6). By contrast, plasma IL-6 levels were not significantly increased during ACS (n=12), compared with vaso-occlusive crisis (n=12), steady state (n=12) and healthy controls (n=9). IL-6 levels were more than 150-fold higher in sputum than in plasma, suggesting increased local production by inflammatory cells during ACS. Sputum levels of IL-8, CCL2 and CCL3 chemokines were also increased during ACS, which may contribute to the recruitment of innate immune cells, such as neutrophils and monocytes, in the lungs. The results strongly suggest an involvement of these inflammatory mediators in ACS pathophysiology and open new therapeutic perspectives, in particular with IL-6 inhibitors.

### FIELD OF THE INVENTION

The present invention is in the field of medicine, in particular haematology.

### BACKGROUND OF THE INVENTION

Sickle cell disease (SCD) is a severe hemoglobin disorder, characterized by hemolytic anemia, recurrent painful vaso-occlusive events and ischemia/reperfusion-driven inflammation.¹ Acute chest syndrome (ACS), a common and potentially life-threatening form of acute lung injury in SCD, is classically defined as fever and/or respiratory symptoms, accompanied by a new pulmonary infiltrate on chest X-ray.² Although ACS is considered a leading cause of morbidity and premature death in SCD patients, underlying pathophysiological mechanisms remain incompletely understood and therapeutic options are therefore limited.³ Multiple factors may contribute to the development of ACS, including viral or bacterial infections, hypoventilation secondary to pain during vaso-occlusive crisis (VOC), fat embolism and thromboembolism, but in most cases, especially in severe forms with acute respiratory failure, pathogenesis remains unclear.^{4,5} A role of inflammation induced by ischemia/reperfusion and hemolysis has been strongly suggested,^{1,6} which may be mediated by the activation of lung endothelium,^{6,7} as well as innate immune cells, including neutrophils, platelets and monocytes.⁸⁻¹⁰ However, the level of pro-inflammatory cytokines and chemokines in the lungs during ACS has not yet been studied. Interestingly, golden sputum, a hallmark of ACS, was shown to be related to an intense exudative process rather than to the presence of bilirubin but its origin remains unknown.¹¹

### SUMMARY OF THE INVENTION

The present invention is defined by the claims. In particular, the present invention relates to the use of IL-6 inhibitors for the treatment of acute chest syndrome in patients suffering from sickle cell disease.

### DETAILED DESCRIPTION OF THE INVENTION

Acute chest syndrome (ACS) is a common and potentially lethal form of acute lung injury in sickle cell disease (SCD). Because pathophysiology remains unclear, therapeutic options are limited to supportive care with empiric antibiotics and red cell transfusion in case of aggravation. A role of inflammation mediated by endothelial and immune cells has been suspected but the levels of pro-inflammatory cytokines and chemokines in the lungs during ACS have not yet been investigated. Here the inventors report dramatically high levels of IL-6, unlike IL-1β and TNF-α, in the sputum from SCD children during ACS (n=12) compared with non-ACS sputum (n=6). By contrast, plasma IL-6 levels were not significantly increased during ACS (n=12), compared with vaso-occlusive crisis (n=12), steady state (n=12) and healthy controls (n=9). IL-6 levels were more than 150-fold higher in sputum than in plasma, suggesting increased local production by inflammatory cells during ACS. Sputum levels of IL-8, CCL2 and CCL3 chemokines were also increased during ACS, which may contribute to the recruitment of innate immune cells, such as neutrophils and monocytes, in the lungs. The results strongly suggest an involvement of these inflammatory mediators in ACS pathophysiology and open new therapeutic perspectives, in particular with IL-6 inhibitors.

Accordingly, the first object of the present invention relates to a method of treating an acute chest syndrome in a patient suffering from sickle cell disease comprising administering to the patient a therapeutically effective amount of an IL-6 inhibitor.

As used herein, the term “sickle cell disease” or “SCD” has its general meaning in the art and refers to a hereditary blood disorder in which red blood cells assume an abnormal, rigid, sickle shape. Sickling of erythrocytes decreases the cells' flexibility and results in a risk of various life-threatening complications. The term includes sickle cell anemia, hemoglobin SC (HbSC) disease, hemoglobin S/beta-thalassemia (HbS/β⁰ and HbS/β⁺) and all the other rare forms of SCD resulting from the interaction of HbS with HbD Punjab, HbO Arab, and Hb Lepore.

As used herein, the term “hemoglobin S” or “HbS” has its general meaning in the art and refers to the mutated beta-globin encoded by the HBB gene. In SCD, the two beta-globin subunits of normal hemoglobin A (HbA) are replaced by two abnormal beta-globin S subunits in the α2β2 Hb tetramer. Typically, the mutation corresponds to E6V mutation wherein the amino acid glutamic acid is replaced with the amino acid valine, which has hydrophobic properties, at position 6 in beta-globin.

As used herein, the term “acute chest syndrome” or “ACS” refers to a frequent cause of acute lung disease in patients (in particular children) with sickle cell disease (SCD). Patients may present with ACS or may develop this complication during the course of a hospitalization for acute vaso-occlusive crises (VOC).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The method of the present invention is particularly suitable for treating patient suffering from a severe acute chest syndrome and who needs artificial respiratory support (e.g. mechanical ventilation).

As used herein, the term “IL-6” refers to human interleukin-6 (UniProtKB: P05231). IL-6 has a wide variety of biological functions in immunity, tissue regeneration, and metabolism. IL-6 binds to IL6R, then the complex associates to the signaling subunit IL6ST/gp130 to trigger the intracellular IL6-signaling pathway. An exemplary amino sequence for IL-6 is represented by SEQ ID NO:1.

SEQ ID NO: 1
>sp|P05231|IL6_HUMAN Interleukin-6 OS = Homo
sapiens OX = 9606 GN = IL6 PE = 1 SV = 1
MNSFSTSAFGPVAFSLGLLLVLPAAFPAPVPPGEDSKDVAAPHRQPLTSS
ERIDKQIRYILDGISALRKETCNKSNMCESSKEALAENNLNLPKMAEKDG
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQM

As used herein, the term “IL-6R” refers to the Interleukin-6 receptor subunit alpha. The term is also known as IL-6 receptor subunit alpha, IL-6R subunit alpha, IL-6R-alpha, IL-6RA, IL-6R1, Membrane glycoprotein 80 (gp80) or CD126. An exemplary amino acid sequence for IL-6R is shown as SEQ ID NO:2. The extracellular domain of IL-6R typically consists of the amino acid sequence that ranges from the amino acid residue at position 20 to the amino acid residue 365 in SEQ ID NO:2.

SEQ ID NO: 2
>sp|P08887|IL6RA_HUMAN Interleukin-6 receptor
subunit alpha OS = Homo sapiens OX = 9606
GN = IL6R PE = 1 SV = 1
MLAVGCALLAALLAAPGAALAPRRCPAQEVARGVLTSLPGDSVTLTCPGV
EPEDNATVHWVLRKPAAGSHPSRWAGMGRRLLLRSVQLHDSGNYSCYRAG
RPAGTVHLLVDVPPEEPQLSCFRKSPLSNVVCEWGPRSTPSLTTKAVLLV
RKFQNSPAEDFQEPCQYSQESQKESCQLAVPEGDSSFYIVSMCVASSVGS
KFSKTQTFQGCGILQPDPPANITVTAVARNPRWLSVTWQDPHSWNSSFYR
LRFELRYRAERSKTFTTWMVKDLQHHCVIHDAWSGLRHVVQLRAQEEFGQ
GEWSEWSPEAMGTPWTESRSPPAENEVSTPMQALTTNKDDDNILFRDSAN
ATSLPVQDSSSVPLPTFLVAGGSLAFGTLLCIAIVLRFKKTWKLRALKEG
KTSMHPPYSLGQLVPERPRPTPVLVPLISPPVSPSSLGSDNTSSHNRPDA
RDPRSPYDISNTDYFFPR

Accordingly, as used herein the term “IL-6 inhibitor” refers to any compound that is able to inhibit the IL-6 signaling pathway. The IL-6 inhibitor to be used in the methods described herein is a molecule that blocks, suppresses, or reduces (including significantly) the biological activity of an IL-6 cytokine, including downstream pathways mediated by IL-6 signaling. Thus the term “IL-6 inhibitor” implies no specific mechanism of biological action whatsoever, and is deemed to expressly include and encompass all possible pharmacological, physiological, and biochemical interactions with an IL-6 cytokine or its receptor whether direct or indirect.

In some embodiments, the IL-6 inhibitor is selected from the group consisting of antibodies directed against the IL-6 cytokine and antibodies directed against the IL-6 receptor (e.g., an antibody specifically binds to IL-6R).

In some embodiments, the anti-IL-6 antibody binds to the amino acid sequence that ranges from the amino acid residue at position 30 to the amino acid residue at position 212 in SEQ ID NO:1.

In some embodiments, the anti-IL-6R inhibitors is an antibody that binds to the amino acid sequence that ranges from the amino acid residue at position 20 to the amino acid residue 365 in SEQ ID NO:2.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibodies are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In some embodiments, the antibody is a humanized antibody. As used herein, the term “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Anti-IL6 or anti-IL-6R antibodies are well known in the art and include those described in Jones S A, Scheller J, Rose-John S. Therapeutic strategies for the clinical blockade of IL-6/gp130 signaling. J Clin Invest. 2011;121(9):3375-3383. doi:10.1172/JCI57158.

In some embodiments, the anti-IL-6 antibody of the present invention is selected from the group consisting of siltuximab, clazakizumab, olokizumab (CDP6038), elsilimomab, and sirukumab.

In some embodiments, the anti-IL-6 antibody is Siltuximab that is a chimeric (human-mouse) monoclonal immunoglobulin G1-kappa antibody produced in a Chinese hamster ovary (CHO) cell line by recombinant DNA technology having a heavy chain as set forth in SEQ ID NO:3 and a light chain as set forth in SEQ ID NO:4.

SEQ ID NO: 3
>Heavy Chain Sequence
EVQLVESGGKLLKPGGSLKLSCAASGFTFSSFAMSWFRQSPEKRLEWVAE
ISSGGSYTYYPDTVTGRFTISRDNAKNTLYLEMSSLRSEDTAMYYCARGL
WGYYALDYWGQGTSVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 4
>Light Chain Sequence
QIVLIQSPAIMSASPGEKVTMTCSASSSVSYMYWYQQKPGSSPRLLIYDT
SNLASGVPVRFSGSGSGTSYSLTISRMEAEDAATYYCQQWSGYPYTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In some embodiments, the anti-IL-6R antibody is tocilizumab, sarilumab, or levilimab (BCD-089).

In some embodiments, the anti-IL-6R antibody is tocilizumab that is a recombinant humanized monoclonal antibody having a heavy chain as set forth in SEQ ID NO:5 and a light chain as set forth in SEQ ID NO:6.

SEQ ID NO: 5
>Tocilizumab heavy chain:
QVQLQESGPGLVRPSQTLSLTCTVSGYSITSDHAWSWVRQPPGRGLEWIG
YISYSGITTYNPSLKSRVTMLRDTSKNQFSLRLSSVTAADTAVYYCARSL
ARTTAMDYWGQGSLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG
SEQ ID NO: 6
>Tocilizumab light chain:
DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLINNFYPREAKVQWKV
DNALQSGNSQESVTEQDSKDSTYSLSSTLTLSKADYEKHKVYACEVTHQG
LSSPVTKSFNRGEC

In some embodiments, the antibody does not comprise an Fc portion that induces antibody dependent cellular cytotoxicity (ADCC). The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ϵ and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD). In some embodiments, the antibody of the present invention does not comprise an Fc domain capable of substantially binding to an FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′)2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. Nos. 6,194,551.

In some embodiments, the IL-6 inhibitor is an inhibitor of an IL-6 cytokine expression or of an IL-6 receptor (i.e. IL-6R) expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of IL-6 or IL-6R mRNA by binding thereto and thus preventing protein translation or increasing mRNA degradation, thus decreasing the level of IL-6 or IL-6R, and their activity, in a cell. For example, antisense oligonucleotides of at least about 15 bases and complementary to unique regions of the mRNA transcript sequence encoding IL-6 or IL-6R can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. IL-6 or IL-6R gene expression can be reduced by contacting a patient or cell with a small double stranded RNA (dsRNA), or a vector or construct causing the production of a small double stranded RNA, such that IL-6 or IL-6R gene expression is specifically inhibited (i.e. RNA interference or RNAi). Antisense oligonucleotides, siRNAs, short hairpin RNAs (shRNAs) and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing IL-6 or IL-6R. Typically, the vector transports the nucleic acid to cells with reduced degradation relative to the extent of degradation that would be observed in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Typically the IL-6 inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils.

The invention will be further illustrated by the following figures and examples. However, these examples and figures should not be interpreted in any way as limiting the scope of the present invention.

### FIGURES

FIG. 1. Plasma and sputum levels of the main pro-inflammatory cytokines and chemokines in children with sickle cell disease (SCD) (A) Plasma level of IL-6 in 9 healthy controls and in 36 SCD patients, in steady state (n=12), during VOC (n=12) and during ACS (n=12). Median [IQR] plasma IL-6 level was not significantly higher during ACS (49 [37-94] pg/ml), compared with VOC (54 [52-72] pg/ml), steady state (36 [27-50] pg/ml) and controls (36 [28-43] pg/ml). (B) By contrast, median IL-6 level in sputum was dramatically elevated (3320 [1233-6459] pg/ml) in SCD patients during ACS (n=12) compared with non-ACS (n=6) respiratory events (13 [11-19] pg/ml, p=0.0009). (C) For the 5 patients who had concomitant sputum and plasma collection during ACS, median IL-6 level was more than 150-fold higher in sputum (6892 [6314-7114] pg/ml) than in plasma (42 [35-48] pg/ml, p=0.0009). (D, F) Median plasma IL-1β and TNF-α levels were not increased during ACS, compared with VOC, steady state and controls. (E,G) Median levels of IL-1β and TNF-α in sputum were not significantly increased during ACS, compared with non-ACS respiratory events. (H-J) Median sputum chemokine levels were higher during ACS compared with non-ACS respiratory events for IL-8 (3632 [1121-11976] pg/ml vs 774 [575-948] pg/ml, p=0.044) (H), CCL2 (591 [263-1624] pg/ml vs 37 [30-46] pg/ml, p=0.0009) (I) and CCL3 (62 [32-105] pg/ml vs 12 [9-19] pg/ml, p=0.01) (J). *p<0.05, ***p<0.001

### EXAMPLE

### Methods

### Study Design

We performed a prospective observational study between 2017 and 2019 in a pediatric French university-hospital SCD reference center. Eligibility criteria were SCD of all types including SS, SC, S/β⁰ and S/β⁺, and age ≥1 year and <18 years. Exclusion criteria were other diseases resulting in increased systemic or pulmonary inflammation (e.g., inflammatory diseases, asthma), and anti-inflammatory treatments. We recruited 42 SCD patients and collected sputum samples during ACS (n=12) or during non-ACS respiratory events (i.e., respiratory symptoms such as cough, tachypnea or hypoventilation during VOC without a new pulmonary infiltrate on chest X-ray) (n=6) and blood samples during ACS (n=12), VOC (n=12), in steady state (n=12) and in controls recruited among unaffected siblings (HbAA) of SCD children (n=9). Sputum was obtained during chest physiotherapy or by endotracheal suctioning for intubated patients. Blood was collected in ethylenediamine tetra-acetic acid, and plasma was obtained by centrifugation (10 min, 3,500 g, 4° C.). Sputum and blood were stored at −80° C. The level of TNF-α, IL-1β, IL-6, IL-8, CCL2 and CCL3 in sputum and in plasma was measured with a multiplex assay (Bio-Plex Pro™ Human Cytokine 27-plex Assay; Bio-Rad), following the manufacturer's instructions. Several clinical and biological data were obtained from the medical files of all patients (Table 1). Informed consent was obtained from parents or legal guardians for all children. The study was approved by a medical ethics committee (GR-Ex/CPP-DC2016-2618/CNIL-MR01).

### Statistical Analysis

Data are expressed as median [interquartile range]. Differences between groups were assessed with Mann-Whitney test or one-way ANOVA with post-hoc test, as appropriate. Paired t-test was used to compare differences between patients for whom IL-6 was measured both in sputum and in plasma, collected concomitantly (n=5). Spearman's rank correlation was used for correlation analyses. Statistical significance threshold was set at a P-value of 0.05.

### Results and Discussion

IL-6 Levels are Dramatically High in the Golden Sputum from SCD Patients During ACS.

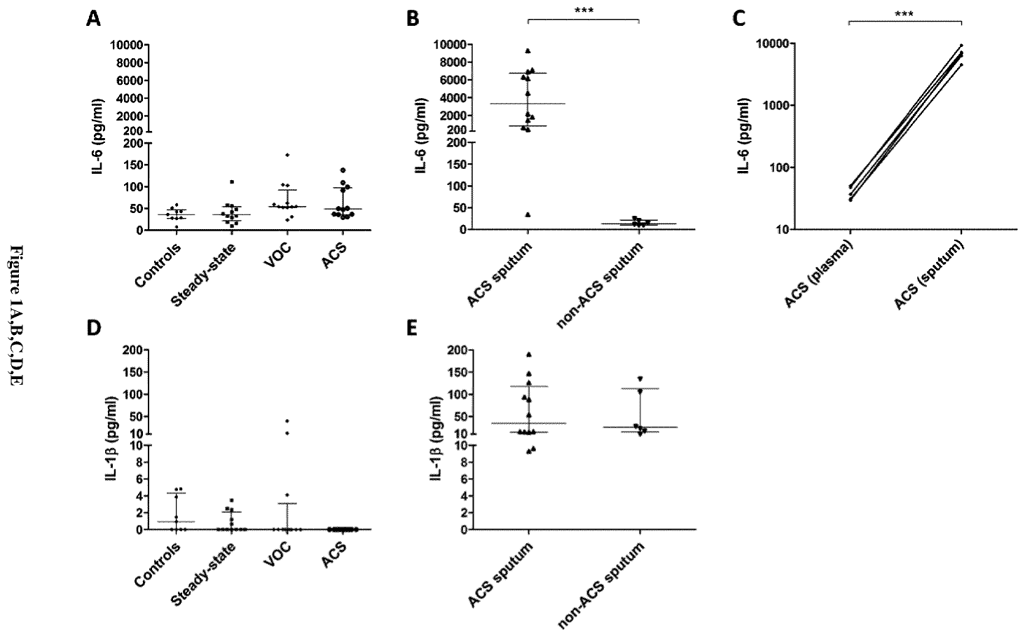

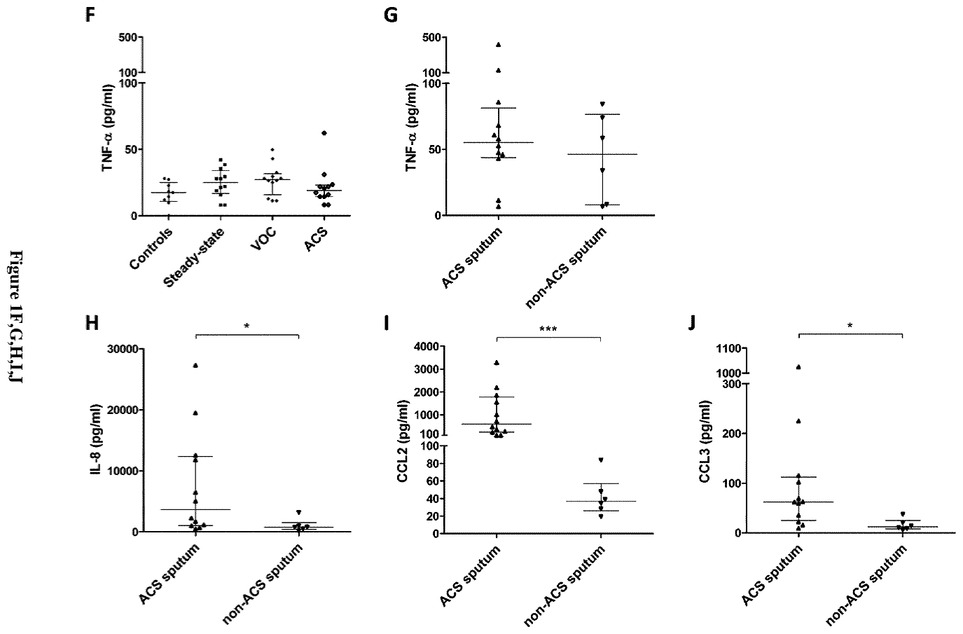

Median [interquartile range] plasma IL-6 level was not significantly increased during ACS (49 [37-94] pg/ml), compared with VOC (54 [52-72] pg/ml), steady state (36 [27-50] pg/ml) and controls (36 [28-43] pg/ml) (FIG. 1A). By contrast, median IL-6 level in sputum was dramatically elevated (3320 [1233-6459] pg/ml) during ACS compared with non-ACS patients (13 [11-19] pg/ml, p=0.0009) (FIG. 1B). For the 5 patients who had concomitant sputum and plasma collection during ACS, median IL-6 level was more than 150-fold higher in sputum (6892 [6314-7114] pg/ml) than in plasma (42 [35-48] pg/ml, p=0.0009) (FIG. 1C). No correlation was found between sputum IL-6 level and CRP level during ACS (r=−0.035, p=0.9).

These results suggest that massive production of IL-6 in the lungs by activated endothelial cells or other inflammatory cells may be involved in ACS pathophysiology, by inducing local inflammation, independently of systemic inflammation. Of note, the five patients with the highest sputum IL-6 levels (>6000 pg/ml) had the most severe ACS forms, two of them requiring invasive mechanical ventilation for respiratory failure, one requiring high oxygen level under non-invasive ventilation, and two presenting multi-organ failure. Therefore, sputum IL-6 level might be a marker of severity in ACS.

An increase in bronchoalveolar IL-6, IL-1β and TNF-α levels has been previously reported in transgenic sickle cell mice exposed to prolonged hypoxia.¹² However, in our patients, no significant increase in the level of IL-1β and TNF-α, was observed in plasma (FIG. 1D and FIG. 1F) and in sputum (FIG. 1E and FIG. 1G) during ACS, suggesting a predominant role of IL-6 in ACS pathophysiology. Sputum IL-6 level is a well-known biomarker of respiratory inflammation, which was recently found increased at steady state in 139 SCD children (mean 43±4 pg/ml) compared with 123 healthy age-matched controls (20±4 pg/ml).¹³

Importantly, a positive correlation was reported between sputum IL-6 level and the number of ACS episodes, supporting our hypothesis of an involvement of IL-6 in ACS pathophysiology. 11-6 is a pleiotropic pro-inflammatory cytokine, involved in the induction of acute-phase responses, which is produced by immune (eg, monocytes, macrophages, neutrophils) and non-immune (eg, endothelial cells, epithelial cells, fibroblasts) cells in response to infectious or non-infectious lung injury. 14 Elevated sputum IL-6 levels in our study are very unlikely to be attributed to viral or bacterial infections because screening for respiratory viruses and atypical bacteria (C pneumoniae and M pneumonia) was negative for all patients, as were blood cultures and bacteriological examination of sputum. Moreover, reported levels of IL-6 in endotracheal fluid from non-SCD children with severe pneumonia requiring mechanical ventilation are 14-fold lower than those observed in our patients.¹⁵

### Sputum Levels of IL-8, CCL2 and CCL3 Chemokines are High During ACS.

Median sputum chemokine levels were higher during ACS compared with non-ACS respiratory events for IL-8 (3632 [1121-11976] pg/ml vs 774 [575-948] pg/ml, p=0.044) (FIG. 1H), CCL2 (591 [263-1624] pg/ml vs 37 [30-46] pg/ml, p=0.0009) (FIG. 1I) and CCL3 (62 [32-105] pg/ml vs 12 [9-19] pg/ml, p=0.01) (FIG. 1J). These high chemokine levels could induce pulmonary recruitment of innate immune cells, including neutrophils and monocytes, which in turn may produce inflammatory mediators, thus contributing to a vicious cycle of local inflammation during ACS. Indeed, in sickle cell mice exposed to prolonged hypoxia, increased neutrophil count was observed in bronchoalveolar lavage together with increased inflammatory mediators and neutrophil lung infiltrates.¹²

Here, we report for the first time markedly increased levels of IL-6, IL-8, CCL2 and CCL3 in the golden sputum from SCD children during ACS. Whether IL-6 should be considered a marker or a full actor of ACS requires further investigations. However, recent reports of dramatic improvement after tocilizumab in a SCD child and an adult patient with ACS related to SARS-CoV-2, suggest that IL-6 may play an essential role in ACS.^{16,17} Clinical trials with immunomodulatory agents targeting IL-6 in patients with severe ACS should be considered.

### Tables:

TABLE 1
Main clinical and biological characteristics of patients with sickle cell disease (SCD) and controls

| | Controls | Steady state | VOC | ACS | Non-ACS |
|---|---|---|---|---|---|
| | (n = 9) | (n = 12) | (n = 12) | (n = 12) | (n = 6) |
| Age (years) | 12.4 [7.9-15.6] | 10.1 [7.9-14.2] | 11.9 [9.6-14.5] | 9.7 [7.2-11.0] | 8.6 [6.1-11.5] |
| Female sex (%) | 67 | 58 | 33 | 33 | 33 |
| SCD type (SS/Sβ⁰/SC) | — | 10/1/1 | 10/1/1 | 12/0/0 | 6/0/0 |
| Hydroxyurea (%) | — | 42 | 75 | 33 | 50 |
| Transfusion program | — | 42 | 0 | 8 | 0 |
| (%) | | | | | |
| Hemoglobin level (g/dl) | 11.7 [11.3-12.1] | 8.8 [8.2-10.3] | 8.3 [7.6-8.8] | 7.1 [6.0-7.8] | 7.6 [7.0-7.8] |
| CRP level (mg/l) | — | — | 22 [15-42] | 149 [134-187] | 41 [21-62] |
| Interval between first | — | — | 1 [1-2] | 2.5 [2-3] | 2 [1-2] |
| symptoms and sample | | | | | |
| collection (days) | | | | | |
| Golden sputum (%) | — | — | — | 100 | 0 |
| Mechanical ventilation | — | — | 0 | 100 (75/25) | 0 |
| (non-invasive/invasive) | | | | | |
| (%) | | | | | |
| Duration of hospital | — | — | 6 [6-8] | 13 [10-16] | 6 [4-8] |
| stay (days) | | | | | |
| Death (%) | — | — | 0 | 0 | 0 |

Data are median [interquartile range] or percentage.
ACS: acute chest syndrome. CRP: C-reactive protein. SCD: sickle cell disease. VOC: vaso-occlusive crisis.

### REFERENCES

Throughout this application, various references describe the state of the art to which this invention pertains. The disclosures of these references are hereby incorporated by reference into the present disclosure.

1. Hebbel R P, Belcher J D, Vercellotti G M. The multifaceted role of ischemia/reperfusion in sickle cell anemia. J Clin Invest. 2020;130(3):1062-1072.

2. Vichinsky E P, Neumayr L D, Earles A N, et al. Causes and outcomes of the acute chest syndrome in sickle cell disease. National Acute Chest Syndrome Study Group. N Engl J Med. 2000;342(25):1855-1865.

3. Miller S T. How I treat acute chest syndrome in children with sickle cell disease. Blood. 2011;117(20):5297-5305.

4. Gladwin M T, Vichinsky E. Pulmonary complications of sickle cell disease. N Engl J Med. 2008;359(21):2254-2265.

5. Jain S, Bakshi N, Krishnamurti L. Acute Chest Syndrome in Children with Sickle Cell Disease. Pediatr Allergy Immunol Pulmonol. 2017;30(4):191-201.

6. Ghosh S, Adisa O A, Chappa P, et al. Extracellular hemin crisis triggers acute chest syndrome in sickle mice. J Clin Invest. 2013;123(11):4809-4820.

7. Belcher J D, Chen C, Nguyen J, et al. Heme triggers TLR4 signaling leading to endothelial cell activation and vaso-occlusion in murine sickle cell disease. Blood. 2014;123(3):377-390.

8. Bennewitz M F, Jimenez M A, Vats R, et al. Lung vaso-occlusion in sickle cell disease mediated by arteriolar neutrophil-platelet microemboli. JCI Insight. 2017;2(1):e89761.

9. Garrido V T, Sonzogni L, Mtatiro S N, Costa F F, Conran N, Thein S L. Association of plasma CD40L with acute chest syndrome in sickle cell anemia. Cytokine. 2017;97:104-107.

10. Allah S, Maciel T T, Hermine O, de Montalembert M. Innate immune cells, major protagonists of sickle cell disease pathophysiology. Haematologica. 2020;105(2):273-283.

11. Contou D, Mekontso Dessap A, Carteaux G, Brun-Buisson C, Maitre B, de Prost N. Golden tracheal secretions and bronchoalveolar fluid during acute chest syndrome in sickle cell disease. Respir Care. 2015;60(4):e73-75.

12. De Franceschi L, Platt O S, Malpeli G, et al. Protective effects of phosphodiesterase-4 (PDE-4) inhibition in the early phase of pulmonary arterial hypertension in transgenic sickle cell mice. FASEB J. 2008;22(6):1849-1860.

13. Al Biltagi M, Bediwy A S, Toema O, Al-Asy H M, Saeed N K. Pulmonary functions in children and adolescents with sickle cell disease. Pediatr Pulmonol. 2020;55(8):2055-2063.

14. Varelias A, Gartlan K H, Kreijveld E, et al. Lung parenchyma-derived IL-6 promotes IL-17A-dependent acute lung injury after allogeneic stem cell transplantation. Blood. 2015;125(15):2435-2444.

15. Nguyen Thi Dieu T, Pham Nhat A, Craig T J, Duong-Quy S. Clinical characteristics and cytokine changes in children with pneumonia requiring mechanical ventilation. J Int Med Res. 2017;45(6): 1805-1817.

16. Odievre M H, de Marcellus C, Ducou Le Pointe H, et al. Dramatic improvement after tocilizumab of severe COVID-19 in a child with sickle cell disease and acute chest syndrome. Am J Hematol. 2020;95(8):E192-E194.

17. De Luna G, Habibi A, Deux J F, et al. Rapid and severe Covid-19 pneumonia with severe acute chest syndrome in a sickle cell patient successfully treated with tocilizumab. Am J Hematol. 2020;95(7):876-878.

1. A method of treating an acute chest syndrome in a patient suffering from sickle cell disease comprising administering to the patient a therapeutically effective amount of an IL-6 inhibitor.

2. The method of claim 1 wherein the patient needs artificial respiratory support.

3. The method of claim 1 wherein the IL-6 inhibitor is selected from the group consisting of antibodies directed against the IL-6 cytokine and antibodies directed against the IL-6 receptor (IL-6R).

4. The method of claim 3 wherein the anti-IL-6 antibody binds to the amino acid sequence that ranges from the amino acid residue at position 30 to the amino acid residue at position 212 in SEQ ID NO:1.

5. The method of claim 4 wherein the anti-IL-6 antibody is selected from the group consisting of siltuximab, clazakizumab, olokizumab (CDP6038), el silimomab, and sirukumab.

6. The method of claim 5 wherein the anti-IL-6 antibody is siltuximab.

7. The method of claim 3 wherein the anti-IL-6R inhibitor is an antibody that binds to the amino acid sequence that ranges from the amino acid residue at position 20 to the amino acid residue 365 in SEQ ID NO:2.

8. The method of claim 7 wherein the anti-IL-6R antibody is tocilizumab, sarilumab, or levilimab (BCD-089).

9. The method of claim 8 wherein the anti-IL-6R antibody is tocilizumab.

10. The method of claim 1 wherein the IL-6 inhibitor is an inhibitor of an IL-6 cytokine expression or of an IL-6 receptor expression.

11. The method of claim 10 wherein the inhibitor of IL-6 cytokine expression or IL-6 receptor expression is an anti-sense oligonucleotides.

12. The method of claim 2, wherein the artificial respiratory support is mechanical ventilation.

13. The method of claim 11, wherein the anti-sense oligonucleotide is an anti-sense RNA molecule or an anti-sense DNA molecule.
